(19) United States
(12) Patent Application Publication
(10) Pub. No.: US 2011/0046888 A1
(43) Pub. Date: Feb. 24, 2011

(54) METHOD OF DIAGNOSING TUMOR DISORDERS IN HUMANS

(76) Inventors: Evgeniya S. Skobeltsina, Kharkov (UA); Boris S. Farber, Brooklyn, NY (US); Alexandr N. Sitenko, Kharkivskoi Oblasti (UA)

Correspondence Address: Adams & Wilks, 17 Battery Place Suite 1231, New York, NY, 10004, US

(21) Appl. No.: 12/450,247

(22) PCT Filed: Aug. 15, 2005
(86) PCT No.: PCT/RU2005/000415

§ 371 Date: Sep. 18, 2009

(51) Int. Cl. G06F 19/00; G01N 33/50

(52) U.S. Cl. 702/19

(57) ABSTRACT

The invention is used for the diagnosis of human tumoral diseases. The diagnosis method consists of preparing films from patient arterial and venous blood, processing said films using a conventional method such as fixing or dyeing, measuring the surface areas of the arterial and venous blood lymphocyte nuclei and comparing said surface areas to corresponding cell surface areas of different lymphocyte groups, plotting regression lines for the arterial and venous blood according to the values thus obtained, determining the percentage thereof for each lymphocyte group, plotting the influence curve thereof in the form of a function of the diameter of corresponding lymphocyte groups, introducing weight factors whose quantity increases along the lymphocyte diameter decrease, multiplying said factors by the lymphocyte percentage, and plotting the sums' correspondence for the arterial and venous blood in the form of the function of the difference therebetween. The diagnosis, location of the tumor, and degree of advancement of the oncological process are jointly determined according to the intersection and correlation of the regression lines and according to characteristic pictures and areas of the curves obtained.

### FIELD OF THE INVENTION

The present invention relates to medicine, namely oncology, and is intended to diagnose tumors in humans.

### BACKGROUND OF THE INVENTION

The method of diagnosing malignant entities is well-known, including the collection of biopsy samples, sample preparation through drying and the introduction of solid residue obtained in the foundation and registration of the infrared absorption spectra, in which native blood serum is used as the biomaterial, and the further recording of the absorption spectrum of the native serum and the relative maximum absorption at different wavelengths to diagnose malignant neoplasms in humans [1].

The disadvantage of the method is its low accuracy rate and complexity of the equipment.

In the method used for diagnosing malignant tumors in humans, the sensitization of blood lymphocytes to tumor antibodies is determined, while in blood smears, the percentage of small lymphocytes is further defined. When this falls to 15% or below in the presence of lymphocyte sensitization, a malignant tumor process is diagnosed.

The disadvantage of the method is its low accuracy rate and ineffectiveness in determining the tumor site [2].

The method of diagnosing cancer in which two samples of blood are prepared, one of which contains the arterial blood and the other of which contains the venous blood, is also widespread. In blood smears, the area of the nuclei of different classes of lymphocytes is determined, as is the area of the particular cells. Using a ratio of cell nuclei area to cell area, regression lines for the relationship of arterial and venous blood are plotted [3].

The disadvantage of the method is the impossibility of determining the tumor site, the extent of the process, and the cancer risk group.

### SUMMARY OF THE INVENTION

The invention's aim is to improve the accuracy of the diagnosis of tumor disorders, determine the options for tumor localization, and assess the spread of the cancer process and the patient's cancer risk group, thereby reducing the physical and psychological impacts of the disease on humans.

The goal is achieved by preparing smears from the patient's arterial and venous blood, treating them in the usual way (fixation, staining), measuring and comparing the area of the nuclei of lymphocytes from the arterial and venous blood with the corresponding areas of cells from different groups of lymphocytes. The data is then used to derive regression lines for the arterial and venous blood for each group of lymphocytes to be created, the lymphocyte percentage is derived and curves are plotted of their dependence as a function of the diameter of the related groups of lymphocytes. Weight coefficients are introduced, the magnitude of which increases with the decreasing size of the lymphocyte diameter. These are multiplied by the percentage of lymphocytes, the dependence of their sum amounts for the arterial and venous blood as a function of their difference are plotted, and the diagnosis and localization of tumor disorders, as well as the degree of the extent of the cancer and the cancer risk patient group is determined in the complex of the cross-section of the superimposed regression lines, as well as by the typical figures and areas of the derived curves.

Regression lines are plotted using the coordinates of the diameter of lymphocytes in microns [μm] (abscissa) and the nuclear-cell ratio (NCR) (ordinate), which is determined by the ratio of the areas of the lymphocytes' nuclei to the areas of the cells. In this method, the points of intersection of the regression line, the axis of ordinates, and the slope are determined and compared. If these are equal, the positioning of the arterial and venous lines of regression and the distances between them are determined; this diagnoses a malignant or benign process. If the slopes are unequal, the point of intersection of the regression curves is used to determine the cancer risk group or the extent of malignancy.

### BRIEF DESCRIPTION OF THE DRAWINGS

The essence of the invention is explained by the following drawings:

FIG. 1: Blood smear (preparation).

FIG. 2: Definition of the area of the nucleus and the cell area.

FIG. 3: Definition of cancer risk with the help of regression lines:

------------ (regression line of venous blood lymphocytes)

______ (regression line of arterial blood lymphocytes).

FIG. 3A: The first risk level.

FIG. 3B: The second risk level.

FIG. 3C: The third (highest) cancer risk level.

FIG. 4: Determination of the extent of the process (disease stages).

FIG. 4A: Stage I of the disease.

FIG. 4B: Stage II of the disease.

FIG. 4C: Stage III-IV of the disease.

FIG. 5: Determination of anticarcinogenic strength in the human benign process.

FIG. 5A-D: Various anticarcinogenic human strengths.

FIG. 6: The percentage of certain groups of lymphocytes in venous and arterial blood in lung cancer (FIG. 6A), uterine cancer (FIG. 6B), and breast cancer (FIG. 6C).

FIG. 7: The percentage of small lymphocytes (A), medium (B), and large (C) in the venous and arterial blood of patients with various tumor sites: lung cancer (FIG. 7A), uterine cancer (FIG. 7B), and breast cancer FIG. 7C).

FIG. 8: Dependence of general result of the multiplication of lymphocyte groups in light of the weight coefficients for arterial and venous blood as a function of the sum of their difference with the confidence interval taken into account.

FIG. 8A: Confidence interval for uterine cancer.

FIG. 8B: Confidence interval for fibromyomas.

FIG. 9: Particular cases of the proposed method of diagnosis.

FIG. 9-1A-1B: patient L.E.

FIG. 9-2A-2B: patient L.N.

### PREFERRED APPLICATIONS

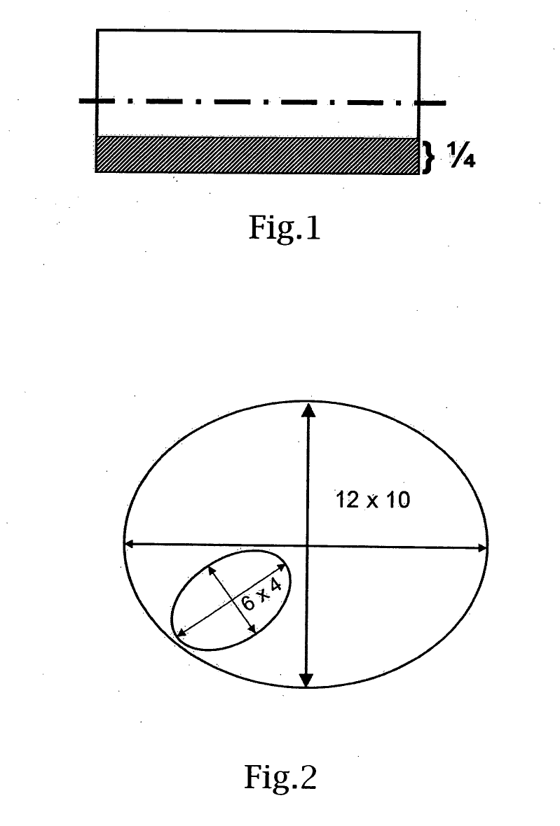

A venous blood sample of 1-2 ml is drawn from the patient's vein, and an arterial blood sample of 3-5 drops is drawn from the finger. Thin (monolayer) smears are prepared. For a reliable picture, symmetry and fast results, it is necessary and sufficient to calculate a ¼ part of the smear area (FIG. 1).
In Leuco- and lymphopenia, 2-3 smears are calculated.
Next, nucleus (An) and cell areas (Ac) are determined (in square micrometers [μm]).
Nucleus area (An) is calculated using the formula:

A nuclear=(½ length of major axis+½ length of minor axis)²×π
Cell area is calculated using a similar formula.
Next, calculate the nucleus area to cell area ratio. For example, in FIG. 2:

An=(3+2)²×π=78.5 μm²

Ac=(6+5)²×π=379.94 μm²

The nuclear-cell ratio (NCR) is equal to 78.5/379.94=0.2066.

These measurements are entered into Table 1.

TABLE 1
Nuclear-cell ratio (NCR) for different lymphocyte groups

| Mean diameter of | Quantity and percentage of | | Nuclear-cell ratio (NCR) of | | Degree of |
|---|---|---|---|---|---|
| lymphocytes in | lymphocytes in the blood from: | | lymphocytes | | lymphocytes' |
| the group(μm) | vein (V) | finger (F) | vein (V) | finger (F) | NCR differences |
| 1 | 2 | 3 | 4 | 5 | 6 |
| 4-4.5 | 0% | 0% | — | — | |
| 4.5-5 | 0% | 0% | — | — | |
| 5-5.5 | 0% | 0% | — | — | |
| 5.5-6 | 0% | 0% | — | — | |
| 6-6.5 | 5-1.11% | 35-7.78% | 0.9500-0.0274 | 0.90314-0.05417 | 0.05 |
| 6.5-7 | 6-1.33% | 35-7.78% | 0.9133-0.0266 | 0.86971-0.05255 | 0.05 |
| 7-7.5 | 43-9.56% | 79-17.56% | 0.9147-0.0457 | 0.86671-0.06168 | 0.001 |
| 7.5-8 | 59-13.11% | 57-12.67% | 0.8795-0.0507 | 0.82333-0.05530 | 0.001 |
| 8-8.5 | 77-17.11% | 67-14.89% | 0.8570-0.0712 | 0.79537-0.07595 | 0.001 |
| 8.5-9 | 38-8.44% | 39-8.67% | 0.8111-0.0892 | 0.75462-0.05991 | 0.001 |
| 9-9.5 | 43-9.56% | 35-7.78% | 0.7588-0.0769 | 0.71457-0.06317 | 0.01 |
| 9.5-10 | 48-10.67% | 32-7.11% | 0.7133-0.0757 | 0.66344 0.06617 | 0.001 |
| 10-10.5 | 59-13.11% | 31-6.89% | 0.6539-0.0577 | 0.62387-0.07805 | 0.05 |
| 10.5-11 | 34-7.56% | 17-3.78% | 0.6215-0.0757 | 0.57647-0.06184 | 0.05 |
| 11-11.5 | 21-4.67% | 15-3.33% | 0.5771-0.0567 | 0.57267-0.04383 | 0.1 |
| 11.5-12 | 6-1.33% | 1-0.22% | 0.5117-0.0397 | 0.61000-0.00000 | 0.1 |
| 12-12.5 | 6-1.33% | 6-1.33 | 0.4967-0.0543 | 0.51167-0.02563 | 0.1 |
| 12.5-13 | 4-0.89% | 1-0.22% | 0.5150-0.0755 | 0.50000-0.00000 | |
| 13-13.5 | 1-0.22% | 0-% | 0.4600-0.000 | — | |
| 13.5-14 | 0-% | 0-% | — | — | |
| 14-14.5 | 0-% | 0-% | — | — | |
| 14.5-15 | 0-% | 0-% | — | — | |

Using linear regression, it is easy to plot regression lines for the arterial and venous blood lymphocytes.

### Regression (Best Fit) Line

The best fit line associated with the n points (x₁, y₁), (x₂, y₂), . . . , (xₙ, yₙ) has the form

$$y = Bx + A$$

$$where$$

$$B slope = m = \frac{n ( \sum xy ) - ( \sum x ) ( \sum y )}{n ( \sum {x}^{2} ) - {( \sum x )}^{2}}$$

$$A y \text{-} intercept = b = \frac{\sum y - m ( \sum x )}{n}$$

$$r = \frac{n \sum ( xy ) - \sum x \sum y}{\sqrt{[ n \sum ( {x}^{2} ) - {( \sum x )}^{2} ] [ n \sum ( {y}^{2} ) - {( \sum y )}^{2} ]}}$$

Here, Σ means “the sum of.” Thus
Σxy=sum of products=x₁y₁+x₂y₂+ . . . +xₙyₙ
Σx=sum of x-values=x₁+x₂+ . . . +xₙ
Σy=sum of y-values=y₁+y₂+ . . . +yₙ
Σx²=sum of squares of x-values=x₁²+x₂²+ . . . +xₙ²
r=regression coefficient
x=lymphocyte diameters
y=Nuclear-cell ratio (NCR) of lymphocytes
Regression Line for arterial blood:

y_{F}=xB_{F}+A_{F}
Regression Line for venous blood:

yᵥ=xBv+Av

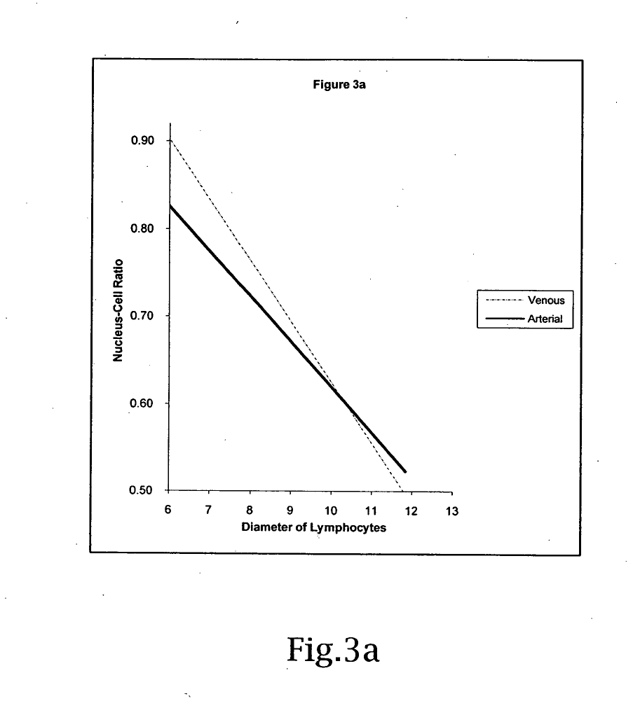

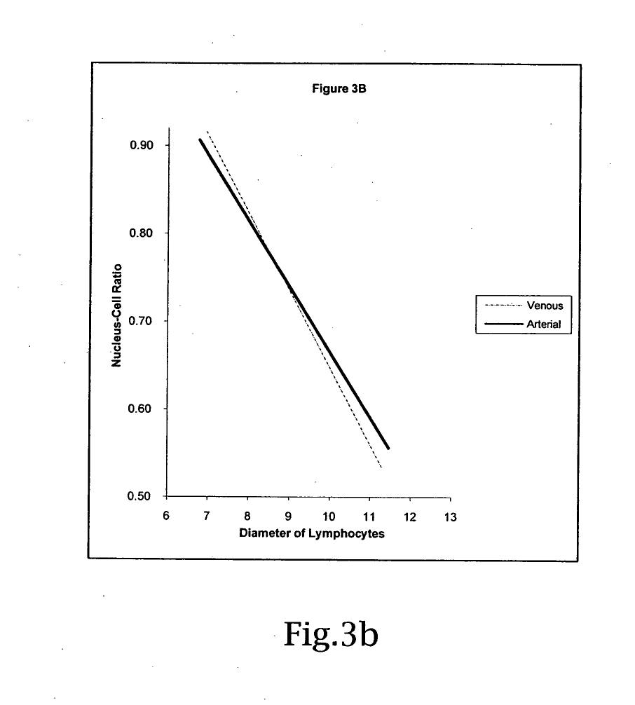

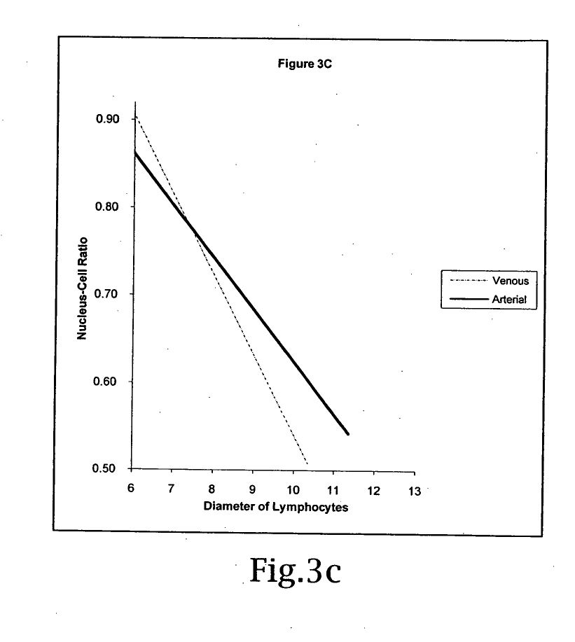

### Identification of Cancer Risk (FIG. 3).

If B_{F}=B_{V}, regression lines are parallel. If B_{F}≠B_{V}, after making the right sides of the equations equal, find the point of intersection of these lines. If the point of intersection (PI) lies in the area of factor space, this decides the patient's risk group. In particular, if the intersection of the regression lines passes through the zone of lymphocytes with a diameter of 11-13 μm, risk level I is indicated. If the intersection of the regression lines passes through the zone of lymphocytes with a diameter of 8-10 μm, risk level II is indicated. If the intersection passes through the zone of lymphocytes with a diameter of 6-7 μm, risk level III (the highest risk level) is indicated.

### FIG. 3A

Regression equation for arterial blood lymphocytes:
Af=1.2126
Bf=−0.0609
r=−0.998
y=1.2126−0.0609x

Regression equation for venous blood lymphocytes:
Av=1.3434
Bv=−0.0729
r=−0.996
y=1.3434−0.0729x

B_{A}≠B_{V}. Find the point of intersection of the regression lines:
1.2126−0.0609x=1.3434−0.0729x
0.0729x−0.0609x=1.3434−1.2126
0.0120x=1.1308
x=10.9

The regression lines intersect at a point where the lymphocyte diameter is equal to 10.9 μM. This patient belongs to the level 1 risk group.

### FIG. 3B

Regression equation for arterial blood lymphocytes:
Af=1.5004
Bf=−0.0836
r=−0.99
y=1.5047−0.0536

Regression equation for venous blood lymphocytes:
Av=1.6496
Bv=−0.1015
r=−0.997
y=1.6496−0.1015x

B_{A}≠B_{V}. Find the point of intersection of the regression lines:
1.5047−0.0836x=1.6496−0.1015x
0.1015x−0.0836x=1.6496−1.5047
0.0179x=1.1449
x=8.095

The regression lines intersect at a point where the lymphocyte diameter is equal to 8.095 μM. This patient belongs to the level 2 risk group.

### FIG. 3C

Regression equation for arterial blood lymphocytes:
Af=1.2705
Bf=−0.0623
r=−0.987
y=1.2705−0.623x

Regression equation for venous blood lymphocytes:
Av=1.3619
Bv=−0.0751
r=−0.995
y=1.3619−0.0751x

B_{A}≠B_{V}. Find the point of intersection of the regression lines:
1.2705−0.0623x=1.3619−0.0751x
0.0751x−0.0623x=1.3619−1.2705
0.0128x=0.0914
x=7.14

The regression lines intersect at a point where the lymphocyte diameter is equal to 7.14 μm. This patient belongs to the level III (highest) risk group.

Areas of risk are defined in patients with a sharp decline in the immune reactivity of the organism: a reduction in T-lymphocyte helpers and a near-complete absence of T-killer lymphocytes.

After appropriate immunomodeling therapy, this situation can be changed; that is, the patient can be withdrawn from the risk zone.

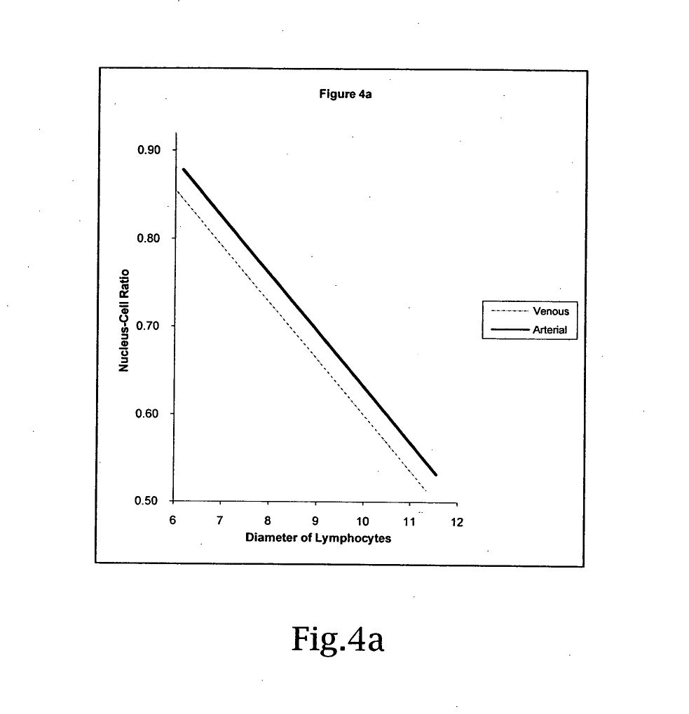

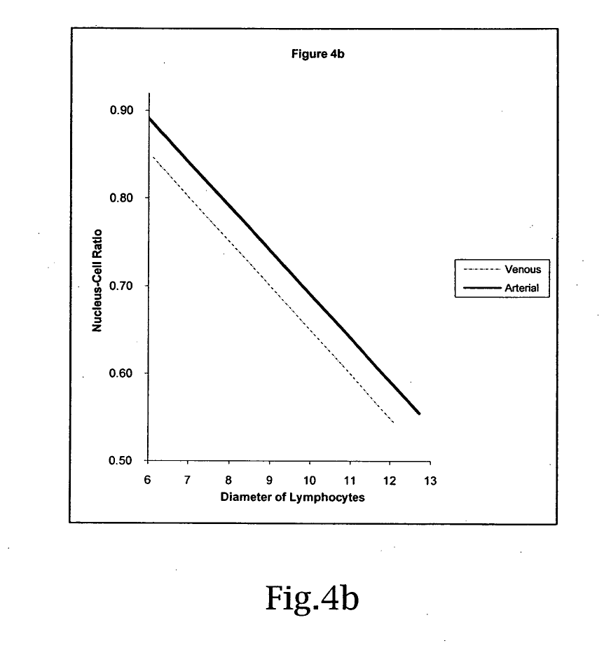

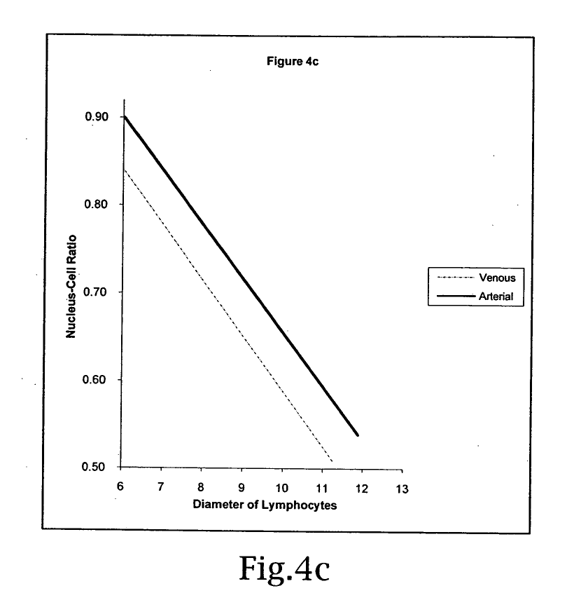

In FIG. 4, the definition of the degree of the extent of the malignant process is presented.

### FIG. 4A

Regression equation for arterial blood lymphocytes:
A_{F}=1.2639
B_{F}=−0.0625
r=−0.99
y=1.2639−0.0625x

Regression equation for venous blood lymphocytes:
Aᵥ=1.2500
Bᵥ=−0.0625
r=−0.992
y=1.250−0.0625x
   BF=Bv The index of the process proliferation is:
A_{F}−Av=1.2639−1.2500=0.0139
0.0139×100=1.39; the patient has stage I breast cancer; lymph nodes are free of metastases.

### FIG. 4B

Regression equation for arterial blood lymphocytes:
Af=1.2705
Bf=−0.0627
r=−0.99
y=1.2705−0.0627x

Regression equation for venous blood lymphocytes:
Av=1.2325
Bv=−0.0627
r=−0.994
y=1.2325−0.0627x
   BF=Bv The index of the process proliferation is:
A_{F}−Av=1.2705−1.2325=0.0380
   0.0380×100=3.8; the patient has stage II breast cancer with lymph node metastases.

### FIG. 4C

Regression equation for arterial blood lymphocytes:
AF=1.2952
BF=−0.0626
r=−0.997
y=1.2952−0.0626x

Regression equation for venous blood lymphocytes:
Av=1.2276
Bv=−0.0610
r=−0.994
y=1.2276−0.0610x
   B_{F}≠B_{V}.

The intersection point of the regression lines is 1.2952−0.0626=1.2276−0.061x. The point of intersection is equal to 42.25, i.e. the lines intersect outside the factor space. In this case, the determination of the extent of the process was conducted as follows:

Define the distance between the regression lines at the points where lymphocyte diameters are equal to 6 and 10 μM.
1.2952−0.0626×6=0.9196 for arterial blood
1.2276−0.0626×6=0.8616 for venous blood
0.9196−0.8616=+0.0580; 0.058×100=5.8, the distance between the regression lines at the point where the lymphocyte diameter is equal to 6 μM
1.2952−0.0626×10=0.6692
1.2276−0.0626×10=0.6176
0.6692−0.6176=0.0516; 0.0516×100=5.16 is the distance between the regression lines at the point where the lymphocyte diameter is 10 μM. Then find the average value: (5.8+5.16)/2=+5.48; the extent of the process in this patient is high at +5.48 (breast cancer with metastases in lymph nodes and lungs, stage III-IV of the disease).

Thus, the higher the index of the process proliferation, the more advanced the stage of the disease is.

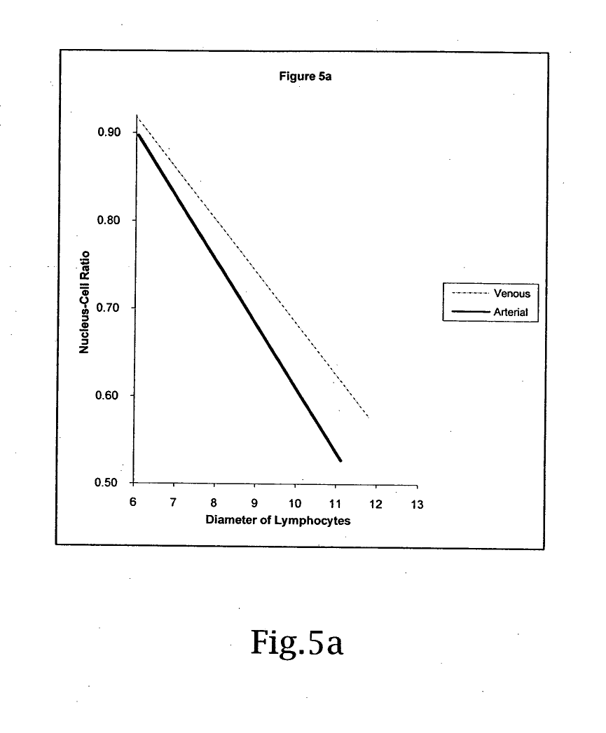

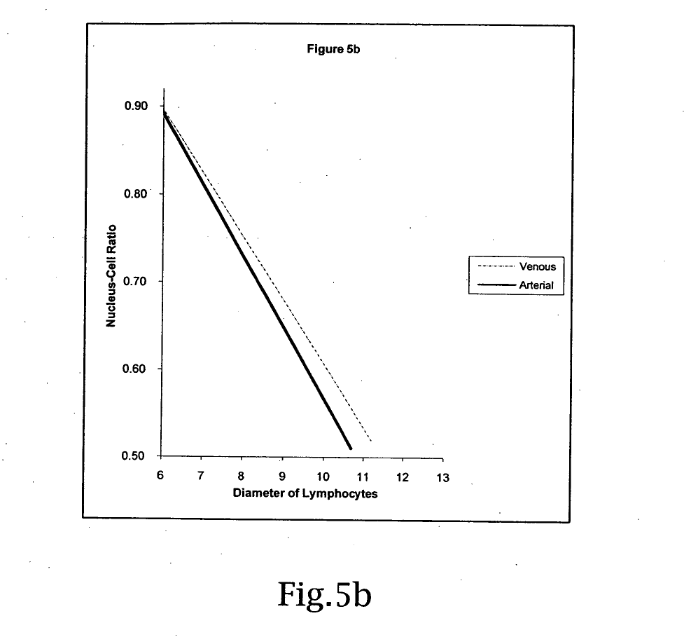

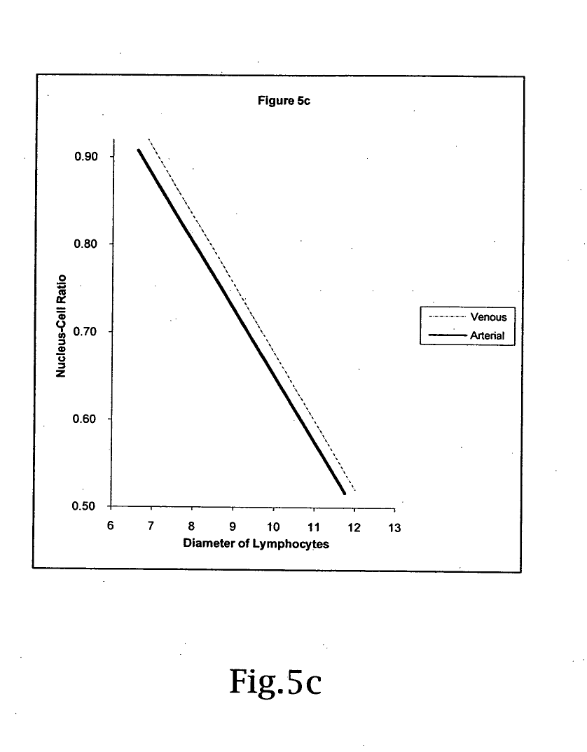

Determination of the anticarcinogenic body strength in the benign process is shown in FIG. 5.

The significance of this indicator is very important for the development of patient treatment methods.

### FIG. 5A

Patient C.E., 40 years old, fibrous mastopathy. Regression equation for arterial blood lymphocytes:
A_{F}=1.342
B_{F}=−0.073
r=−0.99
y=1342−0073x

Regression equation for venous blood lymphocytes:
Av=1.309
Bv=−0.059
r=−0.992
y=1.309−0.059x
BF≠Bv; the regression lines are not parallel.
Calculate the intersection point (IP) of these lines:
1.342−0.073x=1.309−0.059x
1.342−1.309=0.073x−0.059x
0.0300=0.014x x=2.36; the lines cross outside the factorial space.

Then calculate the anticarcinogenic strength of the patient in the regression equation y=A+Bx, where x is 6 and 10 (the diameters of lymphocytes):
1.342−0.073×6=0.9040 (arterial blood)
1.309−0.059×6=0.9550 (venous blood)
Find the difference: 0.9040−0.9550=−0.051
   0.051×100=−5.1
      1.342−0.073×10=0.6120
      1.309−0.059×10=0.7190
      0.6120−0.7190=−0.1070
      −0.1070×100=−10.7; the minus sign (−) indicates the absence of malignant growth.
      −5.1+(−10.7)=−15.8; −15.8/2=−7.9; this gives an absolute value of 7.9, which indicates that this, the anticarcinogenic strength of the patient, is quite high (average: 5÷12).

### FIG. 5B

Patient K.T., 40 years old, ovarian tumor.

Equations of regression lines:
y=1.2401−0.0676x (r=−0.993) (arterial blood)
y=1.3192−0.0702x (r=−0.0998) (venous blood)

Bart≠Bven. Using the abovementioned method of finding the regression lines' intersection point, we get:

1.2401−1.3192=0.0676x−0.0702x

### IP=30.42

Consequently, the lines intersect outside the factor space: lymphocytes with a diameter of 30.4 μM do not exist.
Calculate the anticarcinogenic strength of the patient: in the equation y=A+B(x); substitute the values 6 and 10 for x.
1.2401−0.0676×6=0.8345: the value of the arterial blood's nuclear-cell ratio for lymphocytes with a diameter of 6 μM.
1.3192−0.0702×6=0.8680: the value of the venous blood's nuclear-cell ratio for lymphocytes with a diameter of 10 μM.
   Find the difference: 0.8345−0.8680=−0.0635
   −|0.0635|×100=−|6.35| is the distance between the regression lines of the arterial and venous blood at the point where the lymphocyte diameter is equal to 6 μM.
1.2401−0.0676×10=0.5641
1.3192−0.0702×10=0.6172
0.5641−0.6172=−0.0531
−|0.05311|×100=−|5.31| is the distance between the arterial and venous blood's regression lines for lymphocytes with a diameter of 10 μM.

The anticarcinogenic strength is equal to:
(−6.35+(−5.31))/2=−|5.83|: the anticarcinogenic strength is greater than the lower normal value, and the minus sign (−) suggests that there is no cancer; the tumor is benign.

### FIG. 5C

Patient U.K., 20 years old, breast cancer.
Regression equation for arterial blood lymphocytes:

y=1.3814−0.0741x(r=−0.993)
Regression equation for venous blood lymphocytes:

y=1.4381−0.0770x(r=−0.991)

B_{F}≠Bᵥ.
The intersection point of the regression lines is

1.3814−0.074x=1.4381−0.0770x

The intersection point of 19.35 lies outside the factor space. The procedure for calculating the anticarcinogenic strength is identical to the previous examples:

1.3814−0.0741×6=0.9368
1.4381−0.077×6=0.9661
0.9368−0.9661=−0.0293
−0.0293×100=−|2.93|
1.3814−0.0741×10=0.6404
1.4381−0.077×10=0.6681
0.6404−0.6681=−0.0277
−0.0277×100=−|2.77|
(−2.93+(−2.77))/2=−|2.85|

This young woman's anticarcinogenic strength is very low.

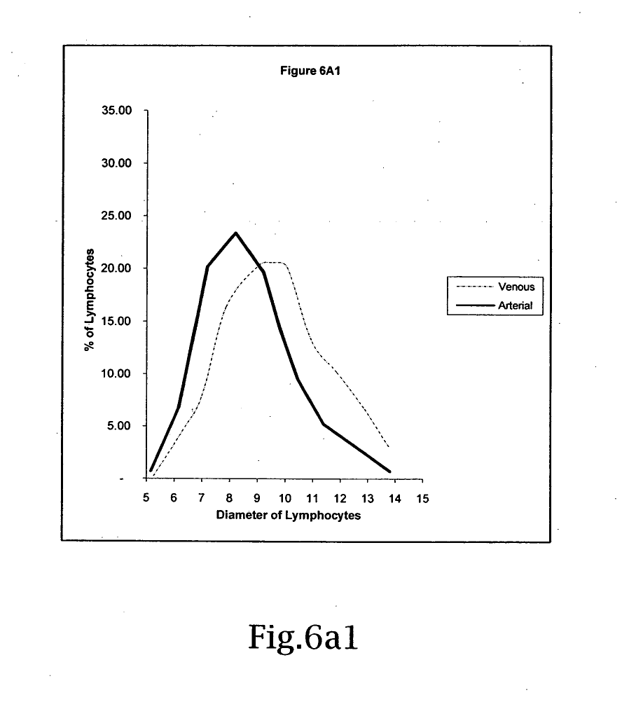

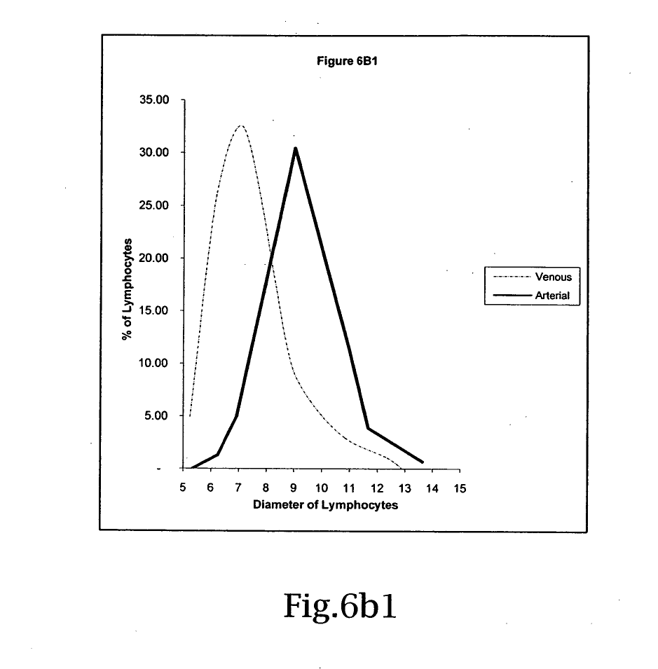

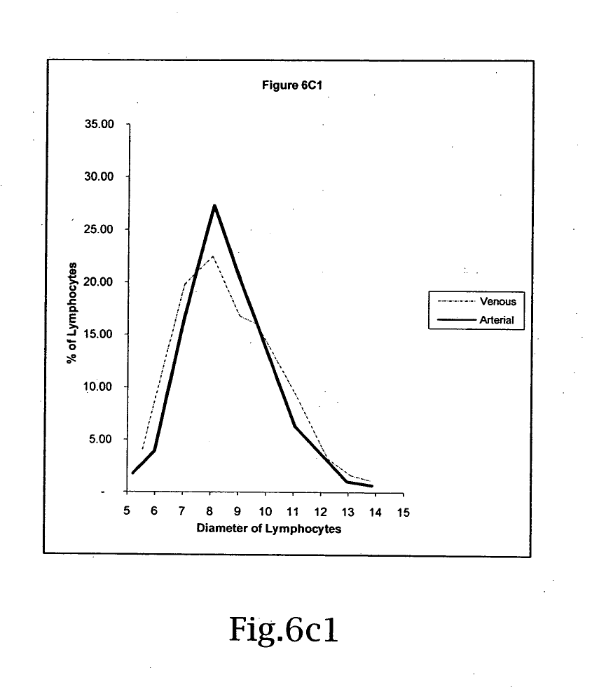

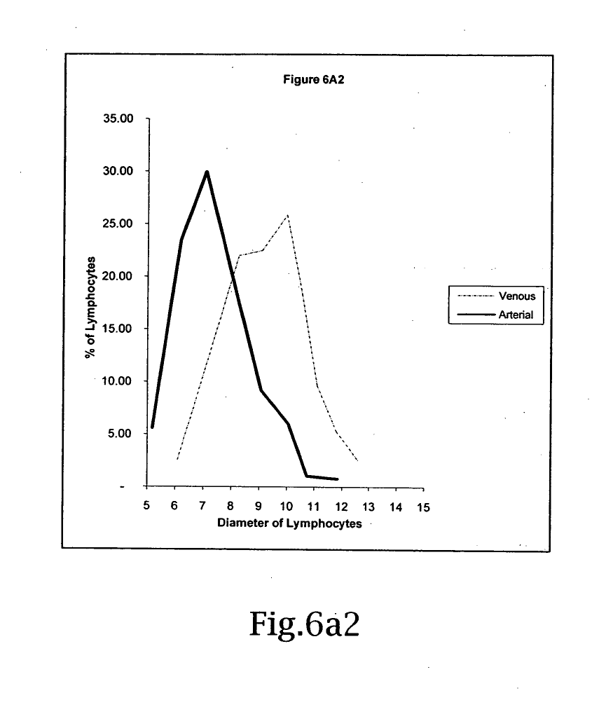

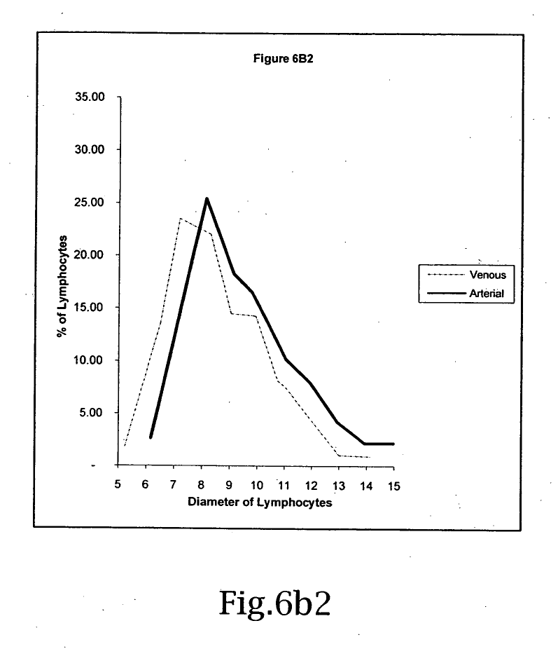

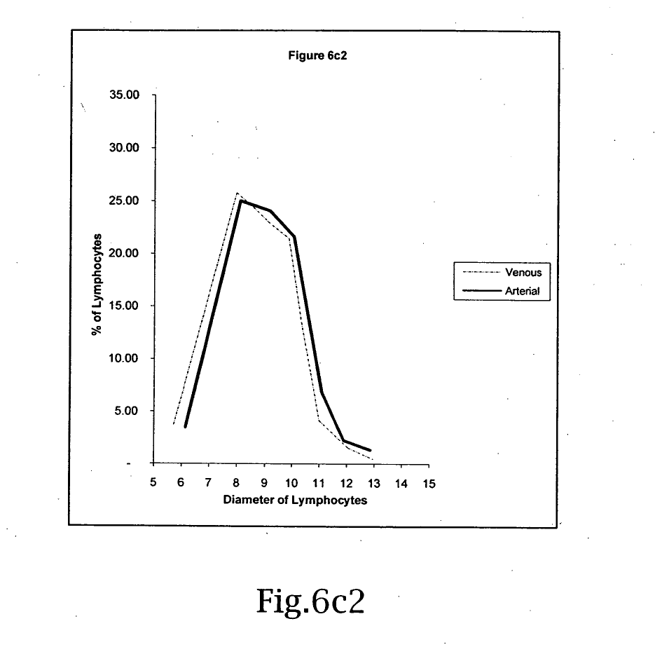

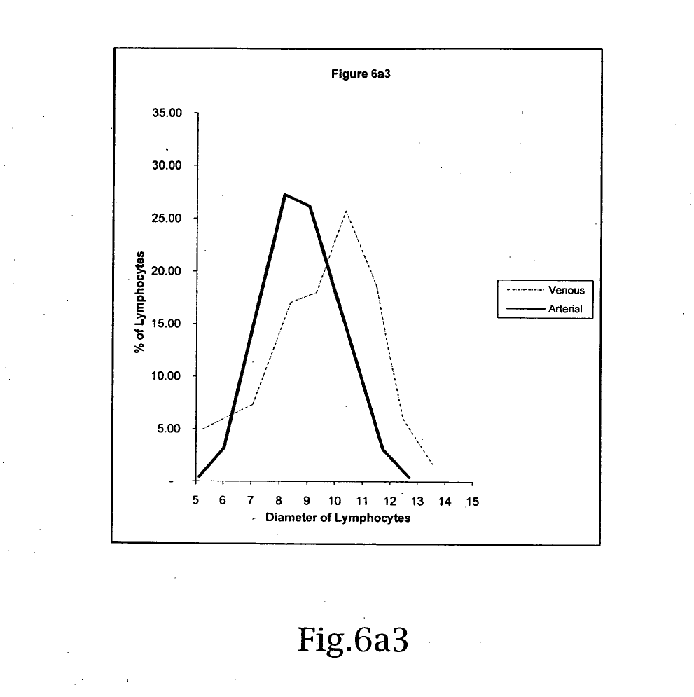

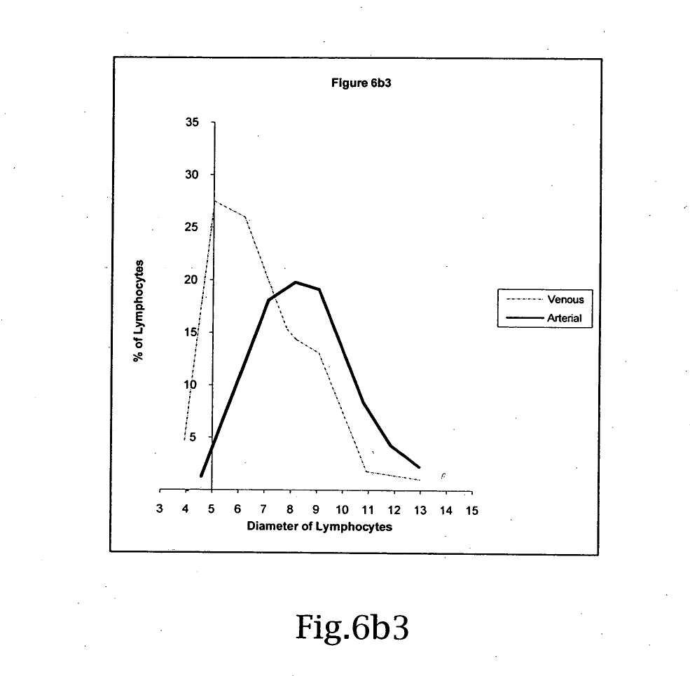

A comparative study of the percentage of certain groups of lymphocytes in the venous and arterial blood at various stages of the malignant process (FIG. 6).

As seen in FIG. 6, graphs of the percentage content of certain groups of lymphocytes in the venous and arterial blood show that the ratio, for example, of small lymphocytes (diameter up to 7.5 μM) in venous and arterial blood in lung cancer (FIG. 6A) differs sharply from the corresponding value in uterine cancer (FIG. 6B) and breast cancer (FIG. 6c).
Create groups of lymphocytes as follows:
Group A: small lymphocytes with a diameter of up to 7.5 μM
Group B: medium-sized lymphocytes with a diameter of up to 10.5 μM,
Group C: large lymphocytes with a diameter of more than 10.5 μM,

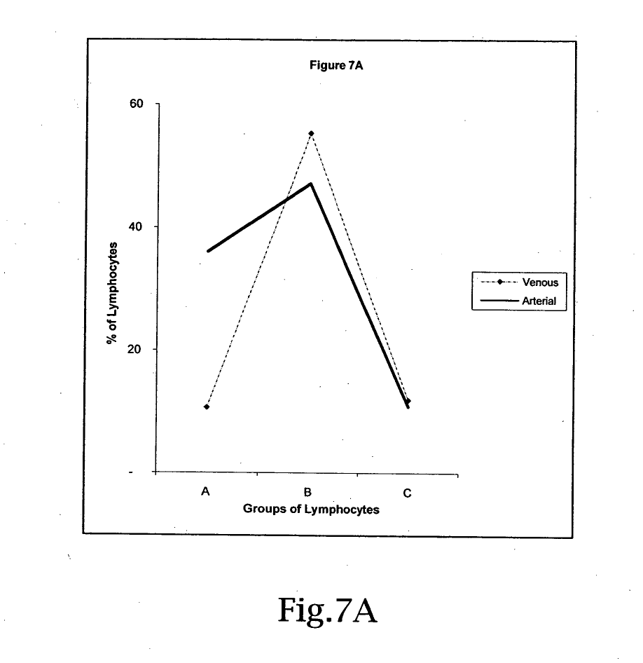

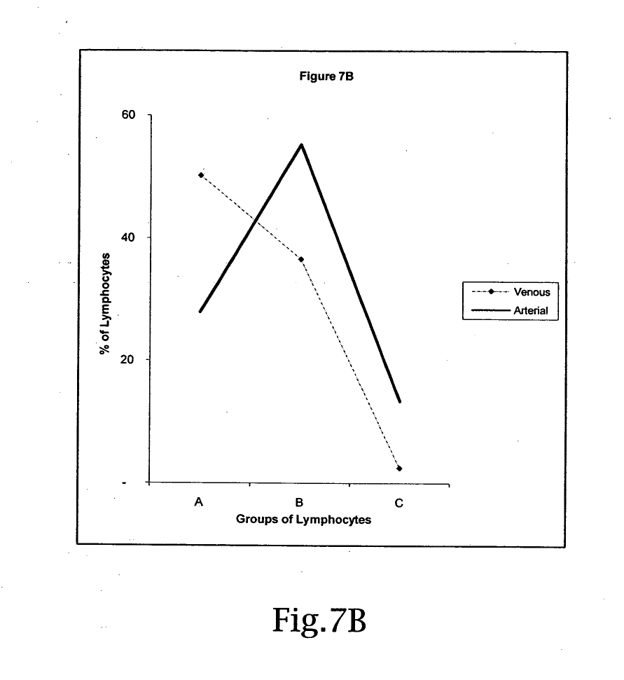

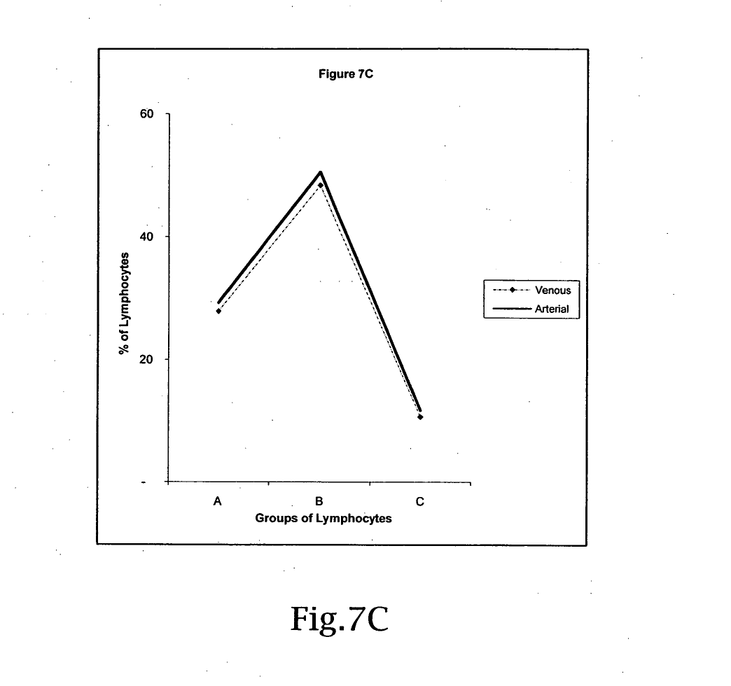

In FIG. 7, we see the results of the percentage of lymphocytes in these groups with lung cancer (FIG. 7A), uterine cancer (FIG. 7B), and breast cancer (FIG. 7C).

### FIG. 7A

Lung cancer: n=38

| Arterial Blood | Venous Blood |
|---|---|
| A = 38.21 ± 10.7 | A = 19.81 ± 10.8 |
| B = 50.3 ± 11.5 | B = 59.7 ± 7.3 |
| C = 10.6 ± 4.0 | C = 19.8 ± 7.4 |

### FIG. 7B

Uterine cancer: n=21

| Arterial Blood | Venous Blood |
|---|---|
| A = 28.66 ± 6.5 | A = 53.61 ± 10.3 |
| B = 58.31 ± 9.2 | B = 40.15 ± 9.2 |
| C = 13.65 ± 4.8 | C = 6.2 ± 3.8 |

### FIG. 7C

Breast cancer: n=19

| Arterial Blood | Venous Blood |
|---|---|
| A = 31.9 ± 7.9 | A = 31.01 ± 9.2 |
| B = 53.72 ± 9.5 | B = 54.7 ± 10.5 |
| C = 11.5 ± 5.02 | C = 11.5 ± 5.02 |

Next, determine the ratio of small lymphocytes to large ones:

1) For lung cancer: arterial blood 38.1/10.6=3.6 19.8/19.8=1 venous blood
2) For uterine cancer: arterial blood 28.66/13.65=2.09 53.61/6.2=8.4 venous blood
3) For breast cancer: arterial blood 31.9/11.5=2.8 31.0/16.3=1.9 venous blood

In calculating these ratios, it is possible to obtain additional data to determine the localization process.

As for improving the accuracy of the diagnosis of tumor disorders, small lymphocytes with a diameter of ≦7 μM are of great importance; to give them a substantial weight, weight coefficients are used. The value of these coefficients should increase with the decrease in lymphocyte diameter.

In particular, we cite the following example. Suppose that the weight coefficients have the structure of aⁿ, where a is the base, and n is power, which can vary and increase with decreasing lymphocyte diameter.

Here is an example using particular patients with tumor processes. In particular, if a=2, we obtain the following results. See Table 2.

After multiplying the percentage of lymphocytes by the corresponding index, we add the sum of the calculations for the arterial blood (ΣF) to the sum of the calculations for the venous blood (ΣV) and divide by 1000 to get the value for the Y axis. Then, from the sum of the calculations for the arterial blood, deduct the sum of the calculations for the venous blood and divide by 1000 to get the value for the X axis.

Getting these values for 20 patients with uterine cancer and 20 patients with fibromyomas, had a regression analysis, where the X axis is −(Σp−Σy)/1000, and the Y axis is −(Σ_{F}+Σᵥ)/1000.

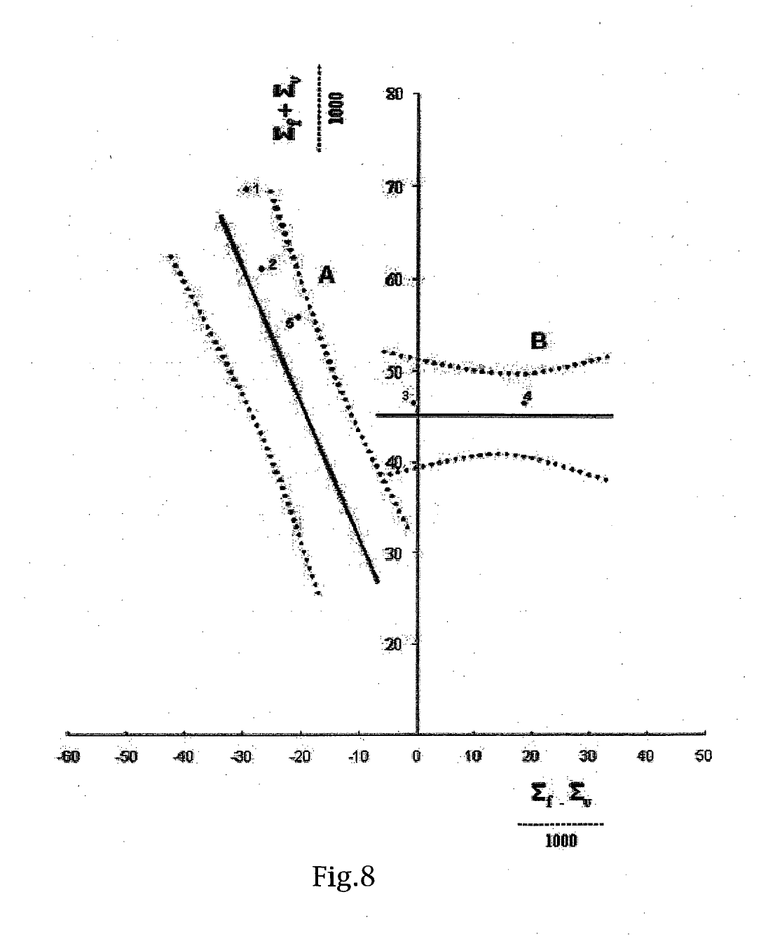

Zone A of FIG. 8 contains patients with uterine cancer; zone B contains patients with fibromyomas. Point 1's coordinates are for patient K.N. (No 1 table. 2) with uterine cancer. Point 2's coordinates are for patient C.T. (No 2 table. 2). Points 3 and 4 from FIG. 8 are for patients with fibromyomas.

Thus it can be seen that these calculations give some additional criteria for the determination of the tumor site.

TABLE 2
Introduction of weight coefficients for different groups of
lymphocytes for the purposes of diagnosis clarification
1) Patient K. N., 28 years old, uterine cancer

| Diameter | % Lymphocytes × | % Lymphocytes × |
|---|---|---|
| Lymphocytes, | index 2ⁿ arterial | index 2ⁿ venous |
| μM | blood lymphocytes | blood lymphocytes |
| 5 | 3% × 1024 (2¹⁰) = 3072 | 28.25 × 1024 = 28928 |
| 6 | 10.25% × 512 (2⁹) = 5248 | 27.25 × 512 = 13952 |
| 7 | 17.25 × 256 (2⁸) = 4416 | 14 × 256 = 3584 |
| 8 | 17.5 × 128 (2⁷) = 2240 | 10 × 128 = 1280 |
| 9 | 16.75 × 64 (2⁶) = −1072 | 9 × 64 = 576 |
| 10 | 16.0 × 32 (2⁵) = 512 | 6.75 × 32 = 216 |
| 11 | 9.25 × 16 (2⁴) = 148 | 3.5 × 16 = 56 |
| 12 | 5.75 × 8 (2³) = 46 | 1 × 8 = 8 |
| 13 | 3 × 4 (2²) = 12 | 0.25 × 4 = 1 |
| 14 | 0.75 × 2 (2¹) = 12 | — |
| | ΣF = 16767.5 | Σv = 48601 |

Σ_{F} + Σᵥ = 65368.5/1000 = 65.3
Σ_{F} − Σᵥ = −31833.5/1000 = −31.8
65.3/−31.8

2) Patient C. T., 46 years old, uterine cancer

| Diameter | % Lymphocytes × | % Lymphocytes × |
|---|---|---|
| Lymphocytes, | index 2ⁿ arterial | index 2ⁿ venous |
| μM | blood lymphocytes | blood lymphocytes |
| 5 | 0.94 × 1024 = 962.56 | 22.75 × 1024 = 23296 |
| 6 | 5.94 × 512 = 3041.28 | 24.83 × 512 = 12712.96 |
| 7 | 22.81 × 256 = 5839.36 | 17.24 × 256 = 4413.44 |
| 8 | 25.31 × 128 = 3239.68 | 11.96 × 128 = 1530.88 |
| 9 | 19.07 × 64 = −1220.48 | 6.9 × 64 = 441.6 |
| 10 | 14.06 × 32 = 449.92 | 9.43 × 32 = 301.76 |
| 11 | 7.81 × 16 = 124.96 | 4.37 × 16 = 69.92 |
| 12 | 2.5 × 8 = 20.0 | 1.84 × 8 = 14.72 |
| 13 | 1.25 × 4 = 5.0 | 0.68 × 4 = 2.72 |
| 14 | 0.31 × 2 = 0.62 | — |
| | ΣF = 14903.86 | Σv = 42784 |

Σ_{F} + Σᵥ = 57687/1000 = 57.6
Σ_{F} − Σᵥ = −27880/1000 = −27.8
57.6/−27.8

3) Patient N. I., 45 years old, fibromyoma

| Diameter | % Lymphocytes × | % Lymphocytes × |
|---|---|---|
| Lymphocytes, | index 2ⁿ arterial | index 2ⁿ venous |
| μM | blood lymphocytes | blood lymphocytes |
| 5 | 3% × 1024 (2¹⁰) = 3072 | 3.92 × 1024 = 4014 |
| 6 | 15.5% × 512 (2⁹) = 7936 | 15.53 × 512 = 7951.36 |
| 7 | 17.5 × 256 (2⁸) = 4480 | 21.07 × 256 = 5393.92 |
| 8 | 22.5 × 128 (2⁷) = 2880 | 20.89 × 128 = 2673.92 |
| 9 | 17.0 × 64 (2⁶) = −1088 | 17.5 × 64 = 1128.96 |
| 10 | 15.2 × 32 (2⁵) = 496 | 12.32 × 32 = 394.24 |
| 11 | 7.0 × 16 (2⁴) = 112 | 7.14 × 16 = 114.24 |
| 12 | 1.5 × 8 (2³) = 12 | 1.25 × 8 = 10.0 |
| 13 | 0.5 × 4 (2²) = 2 | 0.38 × 4 = 1.52 |
| 14 | | |
| | ΣF = 20078 | Σv = 21682.16 |

Σ_{F} + Σᵥ = 41760.16/1000 = 41.7
Σ_{F} − Σᵥ = −1604.16/1000 = −1.6
41.7/−1.6

4) Patient L. E., 40 years old, fibromyoma

| Diameter | % Lymphocytes × | % Lymphocytes × |
|---|---|---|
| Lymphocytes, | index 2ⁿ arterial | index 2ⁿ venous |
| μM | blood lymphocytes | blood lymphocytes |
| 5 | 4% × 1024 (2¹⁰) = 4096 | 0.4 × 1024 = 409.6 |
| 6 | 33.4% × 512 (2⁹) = 17100.8 | 4.8-512 = 2457.6 |
| 7 | 25.6 × 256 (2⁸) = 6553.6 | 15 × 256 = 3840.0 |
| 8 | 16.4 × 128 (2⁷) = 2099.2 | 18.8 × 128 = 2406.4 |
| 9 | 9.2 × 64 (2⁶) = −588.8 | 21.4 × 64 = 1369.6 |
| 10 | 5.4 × 32 (2⁵) = 172.8 | 21.4 × 32 = 684.8 |
| 11 | 3.0 × 16 (2⁴) = 48 | 12.0 × 16 = 192.0 |
| 12 | 2.8 × 8 (2³) = 22.4 | 5.2 × 8 = 41.6 |
| 13 | 0.2 × 4 (2²) = 0.8 | 1.0 × 4 = 4.0 |
| 14 | | |
| | ΣF = 30682.4 | Σv = 11405.6 |

Σ_{F} + Σᵥ = 42088/1000 = 42.08
Σ_{F} − Σᵥ = 19276.8/1000 = 19.27
42.08/19.27

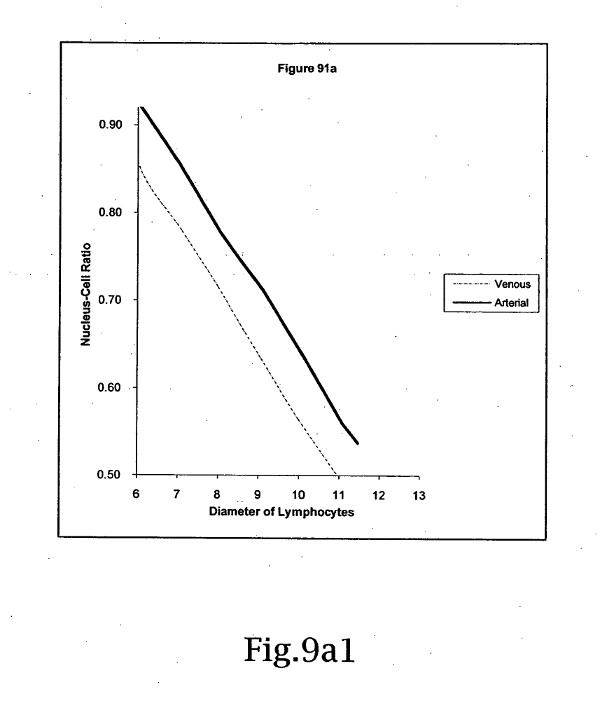

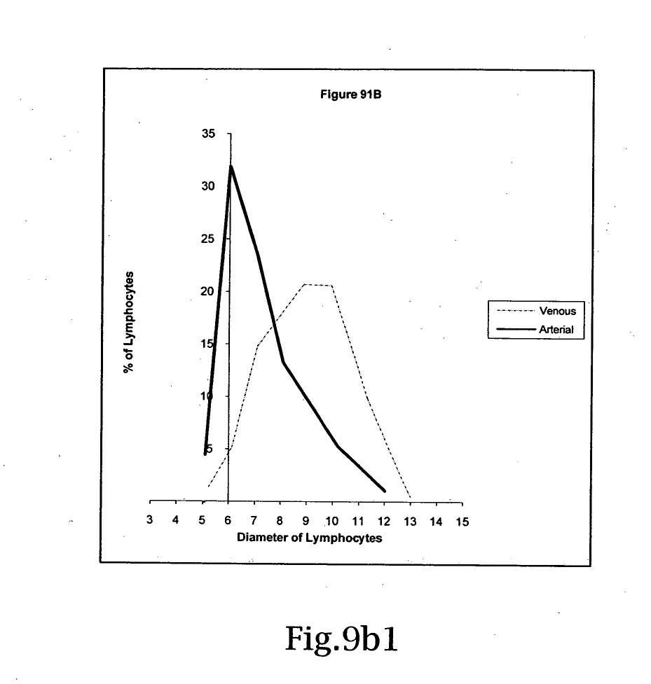

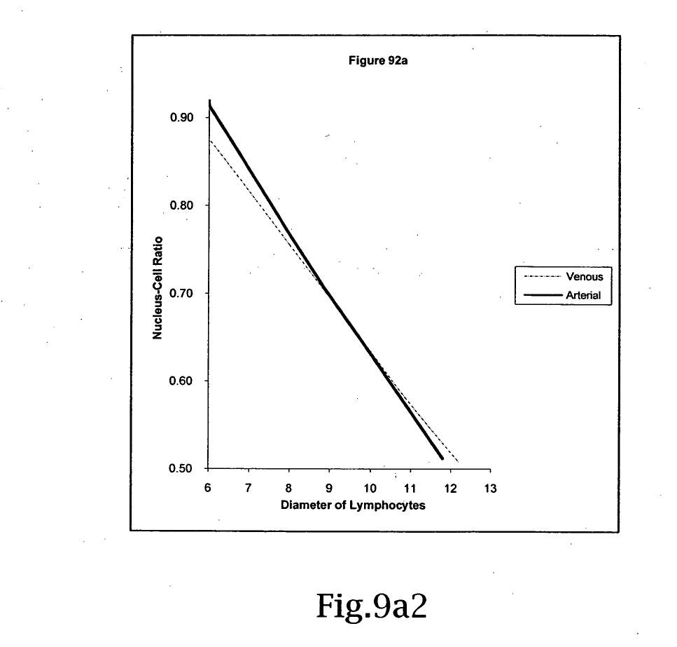

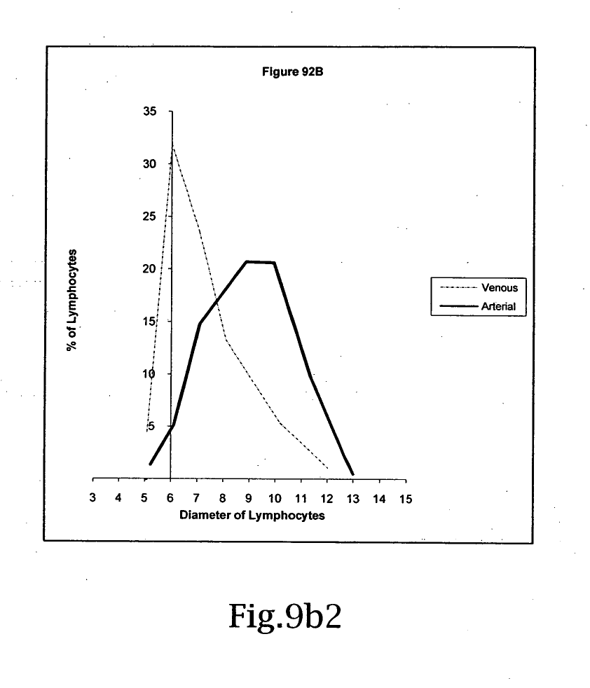

### Examples of Private Cases Diagnosed Using the Proposed Method (FIG. 9)

1. Patient L.E., 50 years old, breast cancer.

Biopsy showed malignant process. The primary diagnosis was stage breast cancer.

The study was performed according to the method suggested here. Results of the regression analysis of the nuclear-cell ratio for arterial (F) and venous blood (V) (FIG. 9IA) show that the process's extent is significant at +6.1.
Calculation of the extent of the process:
Regression equation for the arterial blood:

y_{F}=1.380−0.074x(r=−0.095)
Regression equation for the venous blood:

yv=1.343−0.077x(r=−0.095)
F₆: 1.380−0.074×6=0.9360
V₆: 1.343−0.077×6=0.8810
0.9360−0.8810=0.0550; 0.0550×100=±5.5
F₁₀: 1.380−0.074×10=0.6400
V₁₀: 1.343−0.077×10=0.5730
0.6400−0.5730=0.0670; 0.0670×10=+6.7
(5.5+6.7)/2=+6.1; this index of the extent of the process is significantly higher than the index typical for stage I or II of the disease (see FIG. 4).

In analyzing the percentage of lymphocytes (FIG. 91B), we see that the picture of the percentage of lymphocytes in the arterial blood is characteristic to that of the lung cancer example (see FIG. 6A). On this basis, additional clinical study of the lungs is suggested. The clinical picture identifies metastases in the lungs. Final diagnosis: stage breast cancer with metastases to the lungs.
2. Patient L.N., 32 years old, initial diagnosis: mastopathy? (FIG. 9G) In the study of this patient's blood using the proposed method, the following regression equation was created:

F: y=1.3395−0.0695x(r=−0.995)

V: y=1.26592−0.06268 (r=−0.996)

The regression line of the arterial blood lymphocytes in the area of the small lymphocytes is greater than the regression line of the venous blood lymphocytes. It determines the presence of cancer in the body (FIG. 9_{2A}).

Determine the extent of the process:

### F6: 1.3395−0.0695×6=0.9225 F₁₀: 1.3395−0.0695×10=0.6445

### V6: 1.26592−0.0628×6=0.8898 V₁₀: 1.26592−0.06268×10=0.6391

(0.9225−0.8898)×100=+3.27 (0.6445−0.6391)×100=+0.59
(3.27+0.59)/2=+1.93; the degree of the extent of the process is +1.93. This is the initial stage of the process.

The initial stage of the cancer process was thus identified in the patient's body.

To determine the localization of the process, consider FIGS. 2B and 9. The percentage of different groups of lymphocytes in the arterial and venous blood is characteristic of uterine cancer (see FIG. 6B). To confirm this assumption, we may apply another method, multiplying the percentage of each group of lymphocytes by the weight coefficient corresponding to the particular group (Table 3).

TABLE 3
The introduction of weight coefficients for various groups of lymphocytes
for the purpose of diagnosis clarification (Patient L. N.)

| | Arterial blood | | | Venous blood | | |
|---|---|---|---|---|---|---|
| Diameter | % | co- | | % | co- | |
| of [symbol] | lymph. | efficient | multipl. | lymph. | efficient | multipl. |
| 5 | 0.25 | 1024 | 256 | 10.89 | 1024 | 11156.56 |
| 6 | 7.5 | 512 | 3840 | 28.57 | 512 | 14627.84 |
| 7 | 19.25 | 256 | 4928 | 28.75 | 256 | 7360 |
| 8 | 28.75 | 128 | 3680 | 14.45 | 128 | 1850.88 |
| 9 | 21.25 | 64 | 1360 | 6.78 | 64 | 433.92 |
| 10 | 15.5 | 32 | 496 | 6.6 | 32 | 211.2 |
| 11 | 5.5 | 16 | 88 | 2.35 | 16 | 37.6 |
| 12 | 2.0 | 8 | 16 | 1.6 | 8 | 12.8 |
| 13 | — | — | — | — | — | — |

Σ_{F} = 14664 Σ_{V} = 35690.0
Σ_{F} + Σᵥ = 50354.6/1000 = 50.3 − y
Σ_{F} − Σᵥ = −21026/1000 = −21.0 − x [symbol]
indicates data missing or illegible when filed

These values correspond to point 5 in FIG. 8, the zone of patients with uterine cancer.

After receipt of these results, the patient was sent to the gynecological clinic. The clinic confirmed the conclusion reached through use of the proposed method: a tumor process taking place in the uterus.

Thus, the method we have proposed to diagnose tumor disorders in humans allows us to answer several questions in the complex. Regression equations answer the question of whether or not cancer is present, define the cancer risk group (FIG. 3), determine the extent of the disease (FIG. 4), and determine the anticarcinogenic strength of the organism (FIG. 5). The form of characteristic curves (FIG. 6, FIG. 7, and FIG. 8) allows the localization of the process to be determined.

### APPLICATIONS OF THE METHOD IN THE INDUSTRY

With well-known instrumentation and software (for example, the MEKOS-D and MEKOS-C1 systems) in conjunction with the appropriate software, it is easy to quickly make all the necessary measurements described above for diagnosis.

The MEKOS equipment systems include the manual/conversational (MEKOS-C) or automated (MEKOS-T 1) microscope, with automatic transfer and product focus, video, and computer. In the composition of products can be applied various types of microscopes (Zeiss, NIKON, Olympus, Leisa, Micros, Motis, LOMO, and others), video cameras (Sony, Hitachi, JVC, SIS, Pulnix, Roper Scientific), and other digital or analog 1CCD or 3CCD cameras with progressive scanning and cooling.

1. The method of diagnosis of tumor disorders, comprising the steps of:
a. the collection of samples of the patient's arterial and venous blood
b. the measurement and comparison of the areas of the nucleus of arterial and venous blood lymphocytes in relation to the areas of related cells in different groups of lymphocytes defined by their size
c. the drawing of regression lines based on the data calculated for arterial and venous blood, which differ in that each lymphocyte group's content percentage is determined by the plotting of dependence curves as a function of the diameter of individual lymphocytes and certain groups of lymphocytes
d. the introduction of weight coefficients, the magnitude of which increases with the decreasing size of the lymphocyte diameter, and which are then multiplied by the percentage of lymphocytes; the dependence of their sum amount for the arterial and venous blood as a function of their difference is then plotted
e. the diagnosis and localization of tumor disorders, as well as the stage of cancer spread and the patient's cancer risk group defined in the complex of intersection and collocation of the regression lines as well as the characteristic designs and zones of the curves obtained.

2. The method based on claim 1 differs in that the regression lines are plotted using the coordinates of the lymphocyte diameter (abscissa)-nuclear-cellular ratio (ordinate) determined by the ratio of the area of the lymphocyte nucleus to the area of the lymphocyte cell.

3. The method based on claim 1 differs in that the points of intersection of the regression lines are defined and compared with the axes of the ordinates and their angular coefficients; in the event of their equality, the positioning of arterial and venous lines of regression and the distance between them are determined and tumor disorders are diagnosed therefrom; in the event of the coefficients' inequality, the point of intersection of the regression curves is determined, which establishes the cancer risk group.

4. The method based on claim 1 differs in that the lymphocyte count is produced from a ¼ width of the smear.
